(19) United States
(12) Patent Application Publication
(10) Pub. No.: US 2003/0208103 A1
(43) Pub. Date: Nov. 6, 2003

(54) Entry port for endoscopes and laparoscopes

(76) Inventors: Elazar Sonnenschein, Beersheva (IL); Minelu Sonnenschein, Meitar (IL)

Correspondence Address: Kevin D. McCarthy, Roach Brown McCarthy & Gruber, P.C., 1620 Liberty Building, 420 Main Street, Buffalo, NY, 14202, US

(21) Appl. No.: 10/426,987

(22) Filed: May 1, 2003

(30) Foreign Application Priority Data
May 2, 2002 (IL) 149463

(51) Int. Cl. A61B 1/00

(52) U.S. Cl. 600/117

(57) ABSTRACT

The invention is a device used to determine the depth of insertion and/or the angle of rotation of an elongated body passing through it. The device of the invention comprises at least one sensing element suitable to gauge the movement of the elongated body. In different embodiments of the invention the sensing element is selected from an optical sensor, a Hall effect sensor, or is a sensing element activated by mechanical friction. The device of the invention is especially suited use with endoscopic devices. In the case where the elongated body is a gastroscope, the device of the invention can be a modified bite block.

### CLAIM OF PRIORITY

This application claims priority to Israeli patent application serial number 149463 filed on May 02, 2002

### FIELD OF THE INVENTION

The present invention relates to endoscopic apparatus. More particularly, the invention relates to an apparatus and a method for determining the exact position of an endoscope inserted inside a body.

### BACKGROUND OF THE INVENTION

A medical endoscope is an instrument used to examine and treat the interior of a canal or hollow lumen in the body. Endoscopes and similar devices are also extensively used in industry for similar purposes. In medical applications, endoscopes are used for diagnostic work as well as surgical applications often saving the need for performing an open operation on a patient. In cases in which the endoscope is introduced through the mouth, it is often used in conjunction with a device called a bite block. A bite block, in essence, is a ring-like device typically made of a plastic or other suitable biocompatable material placed in the patient's mouth, between the teeth, through which the endoscope is inserted into the patient's esophagus. The bite block serves to keep the teeth of the patient from clamping on the endoscope and allows easy insertion and withdrawal of the instrument especially in the not infrequent situations in which the patient is unconscious or under anesthesia. The bite block also keeps the endoscope steady for the operator, allowing delicate and intricate proceedures to be carried out.

In prior methods of using an endoscope, operators rely on various means for approximating the location of the endoscope inside the body. By employing a camera that is mounted on the distal tip of the endoscope, the operator can observe the inside of the body, and thus move the endoscope to the required location. Another method is to use lines marked on the proximal end of the endoscope outside the body. By observing the position of these lines relative to a fixed point on the outside, the operator is able to measure how deep the endoscope is inside the body.

Several difficulties arise in relying on these methods for measuring the location of the endoscope:

These methods give an indication about the general area where the endoscope is located, but they lack the accuracy needed for many applications.

It is difficult for the operator to determine how deep the endoscope is, with a single glance. Reading of the lines on the endoscope may depend on the angle of observation and require the operator to constantly shift his attention from his main task.

The lines do not give the operator a continuous reading representing the distance and it is necessary to interpolate between the markings on the endoscope to get intermediate measurements.

Use of a camera to determine the endoscope's location inside the body is accompanied be various problems. For example, the camera lens may become obscured or coated by various internal tissues or fluids;

No method of the prior art allows measurement of the angle of rotation of the endoscope inside the body with good accuracy. The option presently available is to create a mark on the bite block, which could serve as a reference point for a visual estimate of the endoscope's angle of rotation, when looking at the endoscope from the outside.

The effort necessary to keep track of the endoscope's exact position may come at the expense of the operator's ability to efficiently and accurately perform the procedure.

An illustrative example of the use of endoscopes, is their use in the treatment of gastroesophageal reflux disease, or GERD. This disease is characterized in abnormal regurgitation of from the stomach into the esophagus, due to a malfunction of a one-way valve at the junction of the esophagus with the stomach. The surgical treatment for this disease is referred to as fundoplication. Surgical fundoplication is a major operation involving wrapping of the fundus of the stomach around the lower esophagus in an attempt to reconstruct the faulty valve. Endoscopically the procedure is carried out by inserting the endoscope through the patient's mouth and through the esophagus into the stomach. The main advantages of using an endoscopic approach in performing fundoplication, are the ability to carry out this procedure without an invasive operation, and that the use of only local anesthesia is required. Furthermore, the cost of the proceedure is less than that of open surgery and the recovery time is usually faster.

Further discussion about this treatment, as well as on the general structure of an endoscope, can be found in International Patent Application WO 01/67964 filed by the same applicant, the description of which is incorporated herein by reference.

In a proceedure like fundoplication, it is crucial for the doctor to position the endoscope at exactly the right position inside the esophagus. The fundus of the stomach has to be stapled at the proper location, which is usually located 4-5 cm above the gastroesphageal junction. Moreover, in some fundoplications, there is a need for stapling at more than one position around the esophagus. Therefore, it is highly desirable to know precisely the angular position of the endoscope inside the patient. A miscalculation in the location of the staples may hinder the chances of the success of the proceedure and cause damage to the patient. Existing methods for determining the location and orientation of the endoscope in a body do not possess the necessary amount of accuracy or ease of operation to allow the routine performance of the endoscopic fundoplication described in the above-referenced publication.

It is an object of the present invention to provide an apparatus and method which permit accurately to measure the length of a tubular body that has passed through a given point as well as its angle of rotation relative to a reference point.

It is a primary object of this invention to provide an apparatus and a method for accurately measuring the length of the endoscope inserted inside a body.

It is another object of this invention to provide an apparatus and a method for accurately measuring the angle of rotation of an endoscope inserted inside a body.

It is yet another object of this invention to provide a method and apparatus for displaying the length of the endoscope inserted inside a body, as well as its angle of rotation, in clear and easy way for the operator to see, in real time.

It is still a further object of this invention to provide a method and apparatus for keeping track of the location measurements of the endoscope in memory, for future reference.

Further purposes and advantages of this invention will appear as the description proceeds.

### SUMMARY OF THE INVENTION

In a first aspect, the present invention is directed towards providing a device for determining the depth of insertion and/or the angle of rotation of an elongated body passing through it. The device comprises at least one sensing element suitable to gauge the movement of the elongated body. The elongated body can be an endoscope.

The sensing element can be activated by mechanical friction or can be an optical sensor or a Hall effect sensor.

The device comprises:

a. an entrance port;

b. a sensor device;

c. a signal analyzing device;

d. a display device;

e. communication elements between said sensor device and said signal analyzing device; and

f. optionally, a data storing device.

The mechanical sensor of the device consists of one of the following:

a. two wheels, one for detecting and measuring longitudinal motion of the elongated body in a direction generally parallel to its longitudinal axis, and one detecting and measuring rotational motion of the elongated body around this axis; or

b. a ball, which measures both longitudinal and rotational movement of the elongated body.

The device can further comprise a spring located behind at least one of the wheels or ball, a micro-switch located behind one or more of the springs, and/or at least one more wheels or balls in the sensor, possibly attached to a spring, that are designed to increase the friction between the elongated body and the wheels or ball which detect and measure its motion.

If the device comprises a Hall effect based sensor, the elongated body comprises one of the following configurations of magnets:

a. The rings of magnets around the elongated body are positioned with a constant spacing between them; or

b. The rings of magnets around the elongated body are placed adjacent to one another, with their poles inverted.

The entrance port of the device of the invention can consist of a bite block and the sensor device can be attached to the bite block with a flexible pipe. The flexible pipe can be a part of the plastic casting of the bite block. The sensor device can be embedded in the bite block and the information of the movements of the elongated body is passed on to a computer by one of the following means:

a. an electrical cable;

b. a wireless transmitter placed on said apparatus and a receiver outside said apparatus; or

c. a fiber optical cable.

The depth of insertion and the angle of rotation of the elongated body can be displayed on a display device and/or saved to a memory.

In another aspect, the present invention is directed towards providing a method for determining the depth of insertion/or and the angle of rotation of an elongated body passing through the entrance port of a device. The method comprises activating, by means of the movement of the elongated body, a sensing element of which the device is comprised.

It should be noted, that the mention of endoscopes in this application is only for the sake of an illustrative example for the capabilities of this invention, and is by no means a limitation for its scope. Also, fundoplication is described as an illustrative but non-limiting example of an application of the device and method of the invention. Other examples of the uses of this invention are, for example, use with other medical devices such as a laparoscope or a colonoscope, or with devices used for reaching the internal areas of machines and non-organic bodies.

All the above and other characteristics and advantages of the invention will be further understood through the following illustrative and non-limitative description of preferred embodiments thereof, with reference to the appended drawings.

### BRIEF DESCRIPTION OF THE DRAWINGS

FIG. 1 schematically illustrates a conventional endoscope;

FIGS. 2A to 2C are schematic cross-sectional views showing different embodiments of the invention using mechanical sensors;

FIG. 3 and FIGS. 4A and 4B are perspective schematic views showing preferred embodiments of the invention for use in introducing an endoscope into a body through a bit block or similar device;

FIGS. 5A and 5B schematically show the basic principles underlying the use of Hall effect sensors in the present invention;

FIG. 5C is a schematic perspective view and FIG. 5D a schematic cross-sectional view showing the arrangement of ring magnets under the outer coating of the endoscope, according to a preferred embodiment of the invention;

FIG. 5E schematically shows the configuration that should be used to obtain quadrature signals using Hall effect sensors;

FIGS. 5F and 5G schematically show the arrangement of ring magnets under the outer coating of the endoscope, according to another embodiment of the invention; and

FIGS. 6A and 6B schematically show a cross-sectional and perspective view illustrating an embodiment of the invention employing an optical sensor.

### DETAILED DESCRIPTION OF PREFERRED EMBODIMENTS

The general embodiment of the invention is a block with a bore in it, which serves as an entrance port and through which an elongated body (interchangeably referred to hereinbelow as a “tubular-shaped device”, a “tubular device”, a“probing device”, or a “tube”) is inserted. On the wall of the bore is located a sensor to detect and measure motion of the tubular-shaped device. Some examples of the types of sensors that are suitable for this purpose are: mechanical sensors, in which objects, such as wheels or balls, are caused to rotate by the force of friction between the object and the outer surface of the tube moving through the bore; optical sensors: and Hall effect sensors, which are based on currents induced by relative motion of a conductor in a magnetic field. Signals from the sensors are transferred to encoders, which translate them into binary codes or electrical pulses, which are then transmitted by electrical wires, fiber optic cable, or a wireless transmitter, to a microprocessor or computer. The computer processes the data to compute the distance or angle traveled, and records and displays the information.

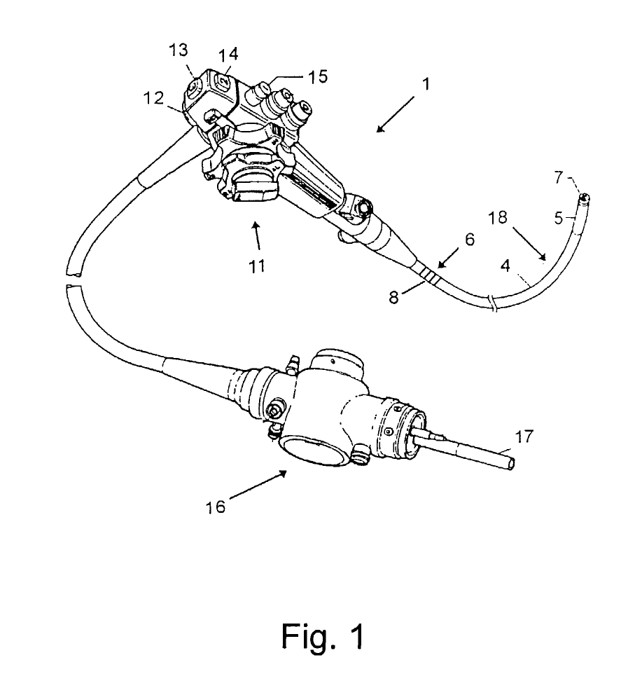

A conventional endoscope is schematically illustrated in FIG. 1. This endoscope comprises several features, such as the operating switches, the angulation lock, etc. that will not be described in detail in the description to follow, because they are conventional, well known to the skilled person and irrelevant to a description of the invention. Briefly the endoscope illustrated in FIG. 1 and generally indicated at 1, is provided with a control section 11 provided with suction valves, locks, switches, etc., switches 12-15 being marked for illustration purposes. It also comprises a connector section 16, used to connect air and water inlets, light guides, etc., the light guide being indicated at 17, for illustration purposes. The insertion tube 18 consists of three separate sections: a flexible portion 4, an articulation section 5 and a distal tip 7.

FIG. 1 can be used to understand how the depth of the endoscope inside a patient's body is measured, according to the prior art. While most of the endoscope is inserted into the patient's body, the proximal part 6 of the flexible portion 4 of the insertion tube 18 remains outside. By observing the position of lines 8 on the outside surface of part 6, relative to a fixed point, for example, a mark on the bite block or the patients teeth, the physician can determine the length of the endoscope that has been inserted inside the patient's body.

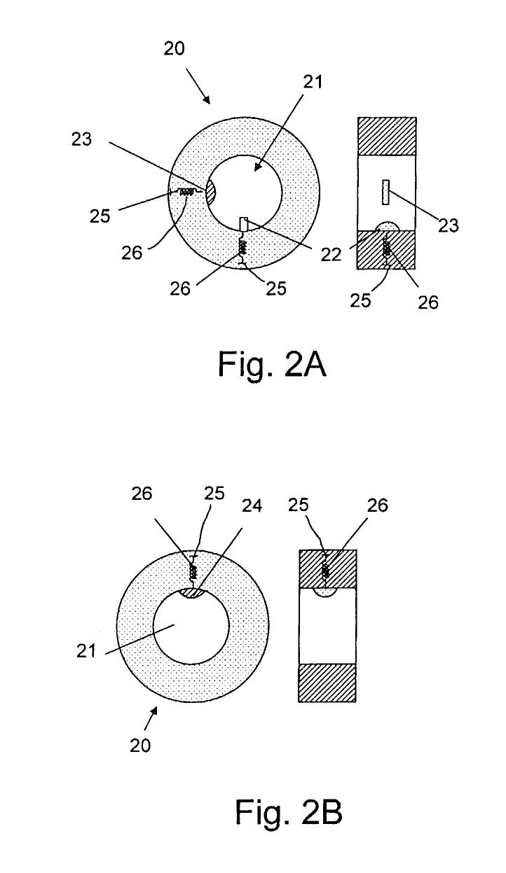

FIGS. 2A to 2C schematically show cross-sectional views of embodiments of the invention employing mechanical sensors. The tubular device is inserted into the object through bore 21 that is in the ring-shaped block 20. In the figures, a cross-sectional view in a plane containing a diameter of the bore is shown on the left, and a cross-section in a plane perpendicular to the above plane is shown on the right.

In the embodiment shown in FIG. 2A, two rotating wheels 22 and 23, arranged to be perpendicular to one another, are mounted on the inside of bore 21. Wheel 22 is situated so that it lies in a plane containing the bore's longitudinal axis, while wheel 23 is situated so that lies in a plane orthogonal to the bore's longitudinal axis.

When the tube is inserted into the bore, it presses on wheels 22 and 23. This causes a micro-switch 25 located inside the wall of the block 20 to be closed calibrating the start of the motion of the tube through the bore of the block. Details of the calibration will be explained in greater detail hereinbelow. As the tubular body moves longitudinally along wheel 22, friction causes wheel 22 to rotate in the direction of motion. Thus, by counting the number of rotations of wheel 22 the longitudinal (insertion) distance of the tube can be determined.

Similarly, as the tube rotates in the bore, friction causes wheel 23 to rotate with it. Thus, the angular distance the tube rotates around its longitudinal axis can be determined.

Each of the wheels 22 and 23 is mounted on an axle. The rotation of the axles is electronically measured using a rotary encoder. Rotary encoders are devices that are capable of converting the rotation of an axle into various types of signals, for example binary, quadrature, or optical signals. The signals are passed on to a microprocessor or computer, which analyzes them to compute how far and in which direction the wheels 22 and 23 traveled and, therefore, the position of the tube relative to the known point of origin. The position of any point on the tube (for example, the distal tip) is then displayed on the computer screen or other display device and/or possibly stored in memory for future reference. The axels of the wheels, rotary encoders, computer, display, and connecting circuitry are not shown in the figures. All of these elements are well known to skilled persons (for example much of this technology is similar to that employed in the familiar “mouse” used with a personal computer) and therefore will not be further described herein.

In the preferred embodiment of the invention shown in FIG. 2B, one ball 24 mounted on the inside of the bore measures both the longitudinal and rotational movements and replaces the wheels 23 and 24 of the embodiment shown in FIG. 2A. In this embodiment, two perpendicular axles are positioned tangential to the ball 24. Friction between the tube and the ball causes the ball to rotate as the tube is moved and friction between the axels and the ball causes them to rotate as the ball rotates. The same technology discussed above with reference to FIG. 2A is used here to measure the rotation of the axles.

In order to make reliable measurements and to avoid slipping of the wheels 22 and 23 (or the ball 24) on the body of the tube, there must be enough friction between them. In a preferred embodiment of the invention, this condition is satisfied by use of a spring 26 located behind each wheel (or ball). The spring 26 presses the wheel (or ball) against the body of the tube while still allowing the tube to be easily moved longitudinally and rotated in the bore. Attached on the opposite end of at least one of thr springs, is the micro-switch 25 described above. When the tube is inserted inside the bore, it pushes on the wheels 22 and 23 (or ball 24), which in turn compress spring 26, closing the micro-switch 25, completing an electrical circuit. The closing of the circuit is used to indicate the origin for the measurements of the motion. In addition, the fact that the circuit remains closed provides an indication of the validity of the readings. A closed circuit indicating that the tube is still exerting sufficient pressure on the springs to mantain the fixed relationship between motion of the tube and rotation of the wheels (ball).

In the embodiment of the invention shown in FIG. 2C, there is an additional supporting ball or wheel 27 with a spring behind it, located also at the inside of the measuring device. Its function is to press the tube tightly against the motion-detecting wheels 22 and 23 (or ball 24). This supporting ball or wheel 27 is not connected to any measuring means, and is only used to further increase the needed friction between the endoscope and the motion-detecting wheels 22 and 23 or ball 24. It can be used as either an alternative to the spring 26 discussed above in reference to FIGS. 2A and 2B, or in addition to it.

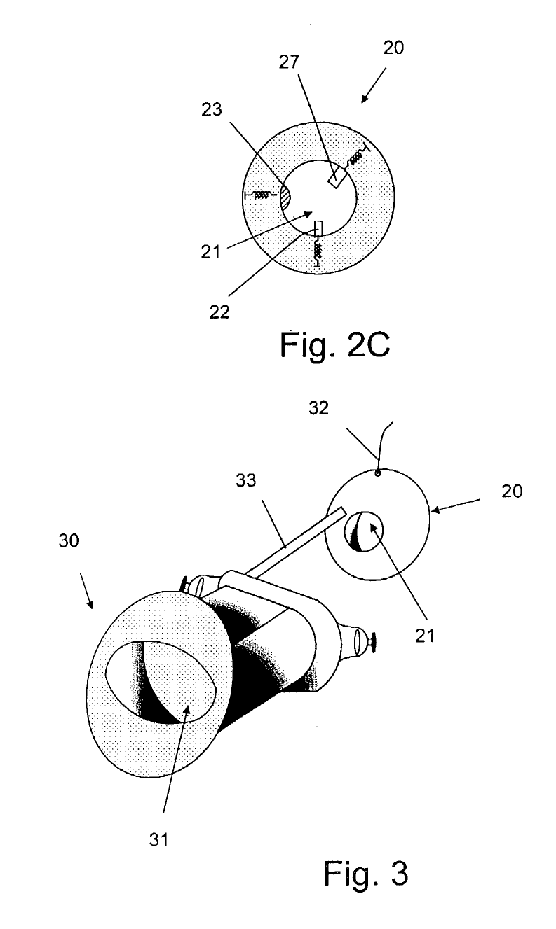

FIG. 3 and FIGS. 4A and 4B are perspective schematic views showing preferred embodiments of the invention for use in introducing an endoscope into a body through a bit block or similar device. In these embodiments, the block with a bore in it 20, which contains the measuring means discussed with reference to FIGS. 2A to 2C, is either separate from the bite block 30 (FIG. 3) or incorporated into the bit block (FIGS. 4A and 4B).

In the embodiment of FIG. 3, the bite block 30 is either a standard bite block commonly used in gastroscopy, or a bite block custom-designed specifically for use with the invention. The endoscope is introduced into the body through bore 21 in the block containing the measurement means and then through the bore 31 in the biteblock, which is clenched between the patients teeth. The block 20 is attached to the bite block 30 with a flexible connection 33 (having optional spring capabilities). This type of attachment gives the operator the freedom to move the endoscope in any direction during the insertion of the endoscope through the bite block. When designed as a custom bite block, the block, flexible connection and bite block are all fabricated together as a single unit, from appropriate material. When using a standard bite block, the flexible connection is designed in such way that it can be attached tightly to both blocks.

In a preferred embodiment of the invention shown in FIGS. 4A and 4B, the measuring means are integrated into the body of the bite block. FIG. 4A corresponds to the embodiment of FIG. 2A and FIG. 4B to that of FIG. 2B.

The output of the sensors is transferred to the computation and display means by means of wire 32. It is possible to replace the wire with wireless connection i.e. a transmitter in the bite block and receiver outside the bite block. The result of the processing of the data is then displayed, in real time, on the computer screen or on any other conventional display unit.

In addition to the mechanical sensors described above, other embodiments of the invention may use other types of motion-detecting sensors. Two examples of such sensors are Hall-effect-based sensors and optical sensors.

As is well known to those skilled in the art, the Hall effect is caused by the deflection of charge carriers moving in a material relative to an applied magnetic field. This deflection results in a measurable potential difference between the sides of the material which is transverse to the magnetic field and the current direction.

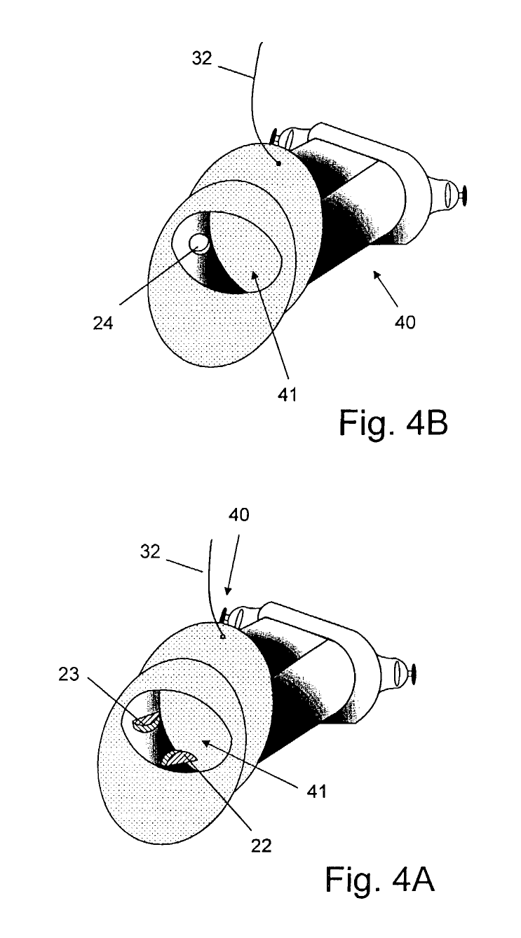

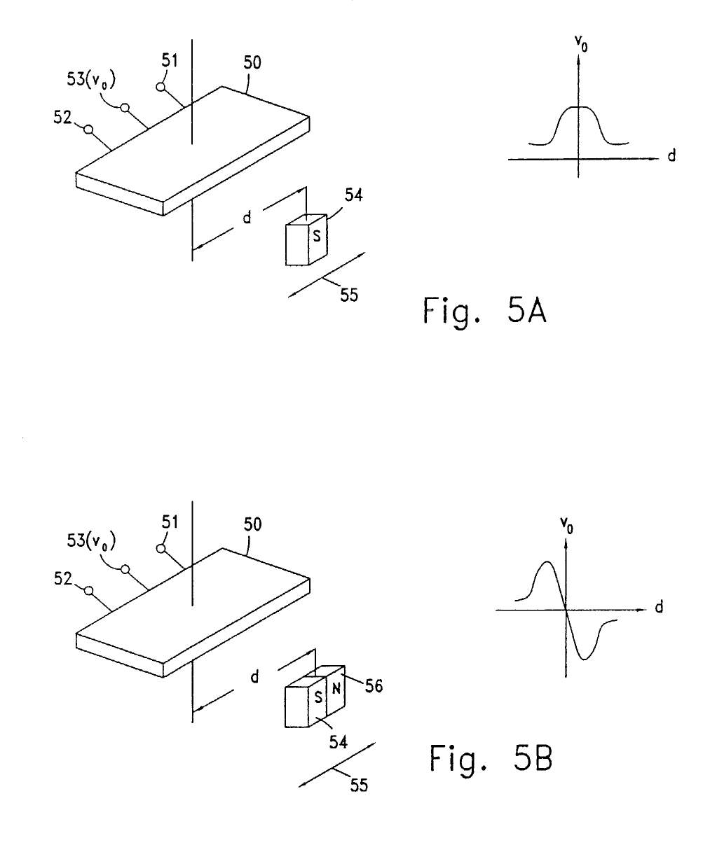

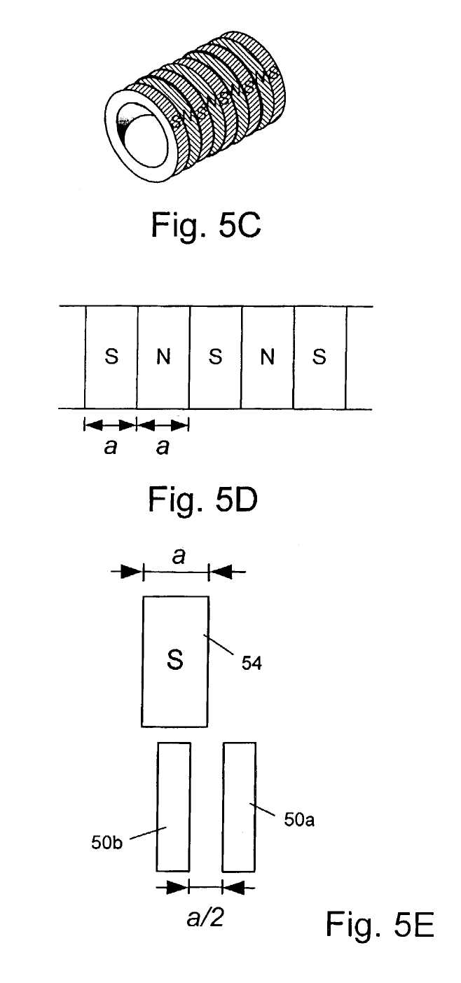

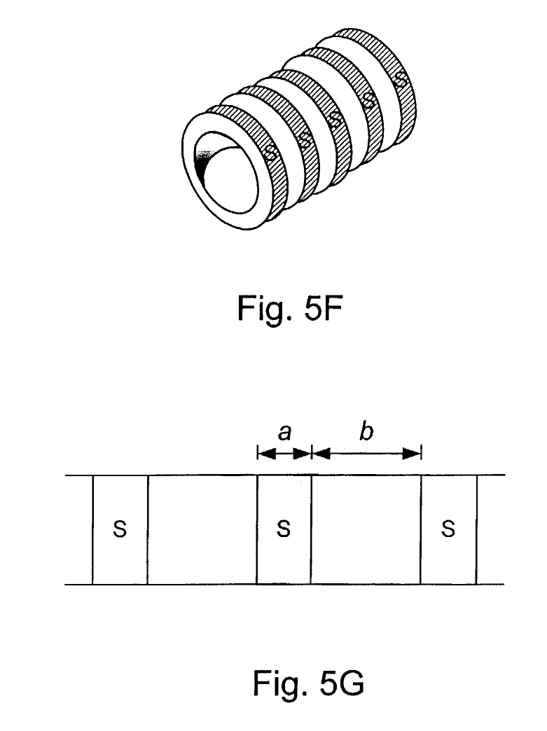

The basic principles underlying the use of Hall effect sensors in the present invention are schematically shown in FIGS. 5A and 5B. Referring to FIG. 5A, sensor 50 is located in a plane and one pole of magnet 54 is located in a parallel plane below the plane containing the sensor. Numerals 51 and 52 designate electrical contacts for the constant current that flows through the sensor and numeral 53 designates the contact at which the output signal (the Hall voltage) is measured. The magnet is moved relative to the sensor such that its pole moves in its original plane in a straight line (indicated in the figure by numeral 55). A line through the center of the sensor and perpendicular to its plane will intersect the line of motion of the pole of the magnet. The distance between the centers of the pole of the magnet and the sensor measured along this line is designated by the letter d. At the far left, (where d is large, the magnetic flux on the sensor is small) there will be essentially no output signal from the sensor. As the motion continues the sensor will start to sense the magnetic field of one of the poles. As the magnet is further moved relative to the sensor a maximum (positive or negative) peak output results, at the point where d=0, corresponding to the highest value of the magnetic flux. As the motion continues to the right the output signal is reduced to zero. The graph on the right shows the output of the sensor v₀ as a function of the distance d between the centers of the sensor and the magnet pole.

FIG. 5B shows the same situation as that of FIG. 5A, with the addition of a second magnet 56, identical to 54, placed next to the first magnet but with its poles reversed. In this case d is measured from the common side of the neighboring magnets and, at d=0, v₀ is also zero.

The magnets can be either permanent or electromagnetic types. Using different numbers of magnets, magnets of different strength, and different configurations of the magnets will lead to different readings and behaviors, from which the distance can be more easily extracted. The skilled person will understand how to adapt the principles discussed with respect to FIGS. 5A and 5B to make changes in the number and/or configuration of the magnets and sensors to enable easier and more accurate determinations of distance and position for specific situations.

In a preferred embodiment of the invention, one or more Hall effect sensors are mounted in the wall of the bite block adjacent to the surface of the bore. On the endoscope, a multitude of ring-shaped magnets are embedded just below its external coating, according to some chosen configuration.

FIG. 5C is a schematic perspective view and FIG. 5D a schematic cross-sectional view, in a plane containing the longitudinal axis of the endoscope, showing the arrangement of ring magnets under the outer coating of the endoscope, according to a preferred embodiment of the invention. In the arrangement shown in the figures, the rings of magnets are placed adjacent to one another with the poles of two adjacent magnets inverted. The magnets are all of equal width a.

In the bite block are mounted two Hall sensors 50a and 50b using the configuration shown in FIG. 5E, i.e., the distance between the sensors is half the width a of the magnets 54. This configuration assures that the signal output from the sensors will be quadrature one to each other and therefore the direction and amount of movement can be extracted. The quadrature wave output of the sensors is handled in similar way to the case of rotary encoders. The resolution of the measurements according to this embodiment is determined by the value of a.

FIGS. 5F and 5G schematically show the arrangement of ring magnets under the outer coating of the endoscope, according to another embodiment of the invention. In this case, the magnets have the same polarity and are placed along the axis of the endoscope with a constant spacing b between adjacent identical ring magnets of width a. In this embodiment, the resolution of the measurement is dependent on b.

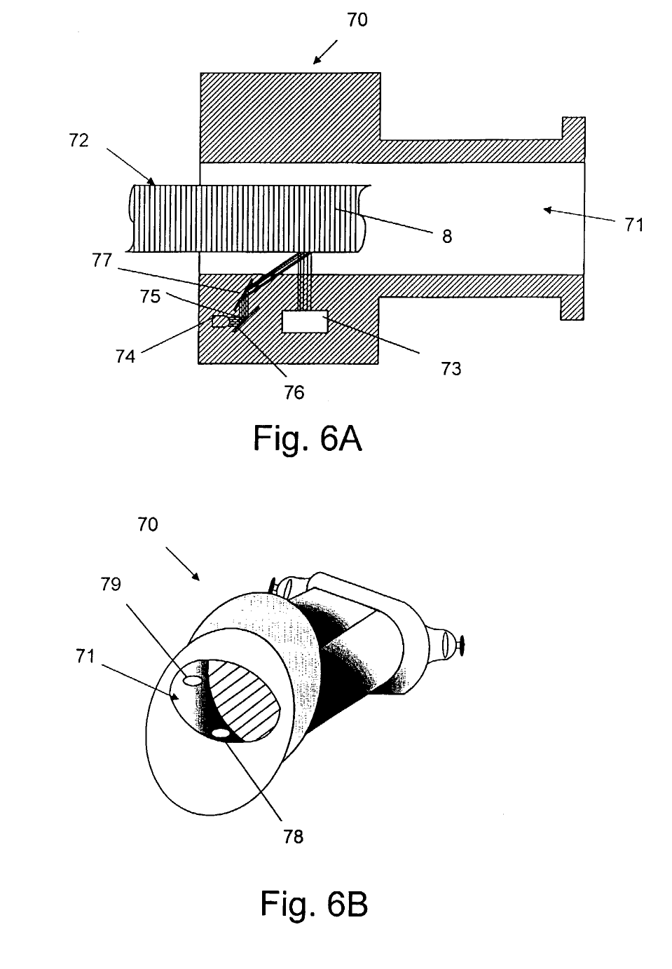

FIGS. 6A and 6B schematically show a cross-sectional and perspective view illustrating an embodiment of the invention employing an optical sensor. This embodiment can be realized using various approaches. A section 72 of an endoscope is shown inserted into the bore 71 in bite block 70. The outer coating of the endoscope has non-reflecting properties. Marked on the non-reflective coating, are special reflective lines 8. The lines 8 can be produced in many ways well known to the skilled person, for example, by printing or painting them on the surface using ink or paint that reflects light having a specific wavelength.

A beam of light 75 is emitted from a LED 74 installed inside the wall of bite block 70, and is directed, by means of mirrors 76, 77, through hollow spaces created in the wall of the bite block until it exits into bore 71 through opening 78. If the endoscope is inserted inside the bore of the bite block, as shown in FIG. 6A, then light beam 75 will encounter its outer surface. If the beam hits the endoscope's non-reflective coating, it will be absorbed. However if the beam hits the reflective lines 78, it will be reflected through aperature 79 and onto an image sensor 73, buried within the wall of the bite block. Movement of the endoscope along the longitudinal axis is thus detected by the image sensor 73. The sensor creates a signal that is transferred to a logic circuit, such as a computer, which deduces the direction and amount of movement based on an image processing analysis of the patterns of the reflected light. The sensor output supports both the PS/2 protocol and quadrature signals, like the output of a rotary encoder.

The use of optical sensors and the methods of analysis of the signals resulting from their use are well known in the art and will not be further described herein. Typical examples of suitable optical sensors that are commercially available are: models HDNS-2000, HDNS-2001, or HDNS-2050 from Agilent Technologies, which are used for optical pointing devices.

The embodiments using Hall effect and optical sensors have described the measurement of longitudinal motion of the endoscope only. The skilled person will have no difficulty in extending the descriptions to include the measurement of rotation around the longitudinal axis of the endoscope.

Although embodiments of the invention have been described by way of illustration, it will be understood that the invention may be carried out with many variations, modifications, and adaptations, without departing from its spirit or exceeding the scope of the claims. For example, the bite block used for gastroscopic procedures can be replaced by a similar entrance port to be affixed at the entrance, either natural or artificial, through which an endoscope or other device is introduced into a body. The skilled person will have no difficulty in making the necessary modifications mutatis mutandis to adapt the methods and apparatus of the invention to any appropriate situation.

1. A device for determining the depth of insertion and/or the angle of rotation of an elongated body passing through it, comprising at least one sensing element suitable to gauge the movement of said elongated body.

2. A device according to claim 1, wherein the sensing element is activated by mechanical friction.

3. A device according to claim 1, wherein the sensing element is selected from an optical sensor and a Hall effect sensor.

4. A device according to claim 1, wherein the elongated body is an endoscope.

5. A device according to claim 1, comprising:
a. an entrance port;
b. a sensor device;
c. a signal analyzing device;
d. a display device;
e. communication elements between said sensor device and said signal analyzing device; and
f. optionally, a data storing device.

6. An device according to claim 5, wherein the sensor device is chosen from the following group:
a. a mechanical sensor;
b. an optical sensor; and
c. a Hall effect sensor.

7. A device according to claim 6, wherein the mechanical sensor consists of one of the following:
a. two wheels, one for detecting and measuring longitudinal motion of the elongated body in a direction generally parallel to its longitudinal axis, and one detecting and measuring rotational motion of said elongated body around this axis; or
b. a ball, which measures both longitudinal and rotational movement of said elongated body.

8. A device according to claim 7, comprising a spring located behind at least one of the wheels or ball.

9. A device according to claim 8, comprising a micro-switch located behind one or more of the springs.

10. A device according to claim 7, further comprising at least one more wheel or ball in the sensor, possibly attached to a spring,; said additional wheel or ball designed to increase the friction between the elongated body and the wheels or ball which detect and measure its motion.

11. A device according to claim 6, comprising a Hall effect based sensor, wherein the elongated body comprises one of the following configurations of magnets:
a. The rings of magnets around said elongated body are positioned with a constant spacing between them; or
b. The rings of magnets around said elongated body are placed adjacent to one another, with their poles inverted.

12. A device according to claim 4, wherein the entrance port consists of a bite block.

13. A device according to claim 12, wherein the sensor device is attached to the bite block with a flexible pipe.

14. A device according to claim 13, wherein the flexible pipe is a part of the plastic casting of the bite block.

15. A device according to claim 12, wherein the sensor device is embedded in the bite block.

16. A device according to claim 1, wherein the information of the movements of the elongated body is passed on to a computer by one of the following means:
a. an electrical cable;
b. a wireless transmitter placed on said apparatus and a receiver outside said apparatus; or
c. a fiber optical cable.

17. A device according to claim 1, wherein the depth of insertion and the angle of rotation of the elongated body are displayed on a display device.

18. A device according to claim 1, wherein the depth of insertion and the angle of rotation of the elongated body are saved to a memory.

19. A method for determining the depth of insertion/or and the angle of rotation of an elongated body passing through the entrance port of a device, comprising activating, by means of the movement of said elongated body, the sensing element of which said device is comprised.

20. A method according to claim 19, wherein the device is a device as claimed in claim 1.
